(19) United States
(12) Patent Application Publication
(10) Pub. No.: US 2021/0030025 A1
(43) Pub. Date: Feb. 4, 2021

(54) BLOOD-FREE DIET FOR REARING MALARIA MOSQUITO VECTORS

(71) Applicants: UNIVERSIDADE NOVA DE LISBOA-INSTITUTO DE HIGIENE E MEDICINA TROPICAL (IHMT-NOVA), Lisbon (PT); CENTRO DE CIENCIAS DO MAR DO ALGARVE, FARO (PT)

(72) Inventors: Henrique SILVEIRA, Lisboa (PT); Maria MARQUES, Lisboa (PT); João CARDOSO, FARO (PT); Rute FÉLIX, FARO (PT); Deborah POWER, FARO (PT)

(21) Appl. No.: 17/046,782

(22) PCT Filed: Apr. 10, 2019
(86) PCT No.: PCT/IB2019/052967

§ 371 Date: Oct. 11, 2020

(30) Foreign Application Priority Data
Apr. 11, 2018 (PT) 110684

(51) Int. Cl. A23K 50/90; A01K 67/033; A23K 20/147; C07K 14/72

(52) U.S. Cl. CPC A23K 50/90; A01K 67/033; A01K 2227/706; C07K 14/723; A23K 20/147

(57) ABSTRACT

The present invention relates to blood-free-diets for malaria mosquito vectors that support ovarian development, egg maturation and fertility, as well as low progeny larval mortality, and normal development of offspring into adult mosquitoes.

The formulated diets comprise supplements of physiological concentrations of several different human GPCR ligands and shown to be an effective artificial meal, free of blood that mimics a vertebrate blood meal and represents an important advance for the sustainability of Anopheles mosquito rearing in captivity.

The present invention is in the domain of genetic engineering, parasitology, entomology medicine, pharmaceuticals and diagnose.

### TECHNICAL DOMAIN OF THE INVENTION

The present invention relates to blood-free-diets for feeding mosquitos, in particular Anopheles mosquitoes vectors of malaria, that are able to support ovarian development, egg maturation and fertility, as well as low progeny larval mortality, and normal development of offspring into adult mosquitoes.

The formulations of the invention may comprise supplements of physiological concentrations of several reagents, namely a protein source such as bovine serum albumin (BSA), ovalbumin, or lactalbumin, a phagostimulant such as adenosine triphosphate (ATP) and cholesterol, and different human GPCR ligands and shown to be an effective artificial meal, free of blood that mimics a vertebrate blood meal and represents an important advance for the sustainability of Anopheles spp. mosquito rearing in captivity.

The present invention is in the domain of genetic engineering, parasitology, medical entomology, vaccine, pharmaceuticals, research and diagnose.

### BACKGROUND OF THE INVENTION

Malaria elimination and the current rise of other vector-borne diseases has led to the development of several innovative mosquito control methods that largely depend on the release into the wild of genetically modified mosquitoes produced in captivity.

Most vector mosquitoes are anautogenous which means that females require a vertebrate blood meal for egg production and development. A major bottleneck for establishing effective mosquito breeding in captivity is that production methods depend on a supply of blood from different animal sources and its quality is often variable.

Current mosquito laboratory rearing strategies use as blood sources sedated or restrained live animals (mice, rats, chickens) or human blood. Nevertheless, foreseeing the urgent need to produce mosquitoes on a large scale, the use of large quantities of blood constitutes a drawback due to ethical concerns and logistical issues associated with demanding safety regulations.

In addition, the mass rearing of mosquitoes requires a specialized animal care facility, qualified personnel, and an efficient feeding system.

These facts might result in a significant hindrance since local regulations, ethical concerns, and infrastructure vary considerably among different countries. Taken together, these limitations are prompting research directed at the development of an artificial meal capable of mimicking blood in terms of producing viable mosquito eggs.

Artificial diets based on the rich nutrient content of human blood have shown to prompt female mosquito oogenesis and fertility in vivo with limited success in comparison to a standard vertebrate blood meal. Successful artificial diets for vector mosquitoes, must fulfil the following requirements: 1) female mosquitoes must fully engorge the meal, 2) the artificial diet must allow vitellogenesis to occur, 3) it must result in large egg batches, and 4) the offspring fitness should be comparable to wild mosquitoes.

Until now, little is known about artificial meals for Anopheles spp. but several studies have been published for Aedes mosquitoes. Alternative vertebrate blood-free meals for Aedes mosquitoes must supply an energy source for mosquitoes to feed (e.g. ATP), a protein source essential for egg maturation, carbohydrates for energy consumption, and amino acids (aa) that are vital for egg production.

In fact, the amino acid composition of the diet is one of the major limiting factors of female mosquito fertility as this depends on the blood source. Besides proteins, a blood meal might also provide lipids, particularly cholesterol for egg production improvement.

Insect reproduction and nutrient metabolism are also regulated by a series of neuropeptides many of which act through the activation of heterotrimeric GTP-binding protein (G protein)-coupled receptors (GPCRs) in the presence of an internal or external stimuli. GPCRs are a large family of cell surface proteins. At least 276 receptor genes exist in the Anopheles genome, many of which are orphans and have currently uncharacterised functions. Conserved sequence homologues of mosquito GPCRs exist in humans and their activating peptides circulate freely in blood and regulate a diversity of physiological functions including energy metabolism and reproduction. This means that during mosquito blood feeding it is likely that interactions between mosquito GPCRs and putative vertebrate peptides may occur.

The insect and human GPCR systems have recently been characterised using comparative genomic approaches. The mosquito genome contains orphan receptor genes that have a similar sequence and may be function homologues of some vertebrate GPCRs. The exposure of mosquitoes to human GPCR-activating peptides when they take a blood-meal raises a number of interesting questions about co-evolution of protein-protein interactions and cross-regulation of physiological systems.

Document WO2018049229 A1 discloses a synthetic blood-free diet formulation for maintenance of hematophagous insects, including Anopheles mosquitoes, said formulation comprising a protein source such as BSA or egg albumin, a carbohydrate source such as glucose, sucrose, fructose, or mixtures thereof, and a lipid source having cholesterol. However, none of these formulations comprise ATP or human/animal GPCR ligands.

Jason Pitts, R. describes a blood-free protein meal supporting oogenesis in Aedes mosquitoes (A blood-free protein meal supporting oogenesis in the Asian tiger mosquito, Aedes albopictus (Skuse). J. Insect Physiol. 64, 1-6 (2014). The formulations described herein comprise BSA dissolved in a saline solution, and ATP dissolved in the BSA solution. However, these formulations do not comprise human/animal GPCR ligands or cholesterol, and for this reason the formulations of Jason Pitts are not suitable enough to provide good rates of oogenesis and egg production, or even on mosquito fitness.

The development of a successful blood substitute diet that allows mass production of anautogenous Anopheles mosquitoes without the need for costly animal care facilities or blood or plasma supply is thus a real need in research and medical fields.

Therefore, the present invention proposes to develop a chemically well-defined artificial blood-free diet to provide a reliable and consistent nutrition to adult mosquitoes that is able to mimic a standardized vertebrate blood meal. The fresh-blood-free diet of the present invention stimulates oogenesis and egg production and has a similar or superior effect on mosquito fitness relative to a standard vertebrate blood.

### SUMMARY OF THE INVENTION

The present invention relates to blood-free-diets for feeding mosquitos, in particular Anopheles mosquitoes vectors of malaria, that are able to support ovarian development, egg maturation and fertility, as well as low progeny larval mortality, and normal development of offspring into adult mosquitoes.

Therefore, in a first aspect of the present invention artificial blood-free diets comprising one or more peptides selected from a listed group of Table 1 for rearing mosquitoes are disclosed according to claim 1.

In a second aspect, the present invention relates to a method of rearing mosquitoes by providing them with the diets of the invention, according to claim 6.

In another aspect, the present invention relates to a process of producing said artificial blood-free diets, according to claim 9.

In another aspect, the present invention relates to a method of rearing mosquitoes by providing them with the diets of the invention, according to claim 11.

In short, the main advantages of the blood-free diets of the invention are:
The availability of a successful blood-free diet is able to allow mass production of anautogenous mosquitoes without the need for costly animal care facilities or blood or plasma supply;
The development of a chemically well-defined artificial diet to provide a reliable and consistent nutrition to adult mosquitoes is a breakthrough for mass rearing of mosquitoes;
The blood-free diet formulated stimulates oogenesis and egg production and has a similar or superior effect on mosquito fitness relative to a standard vertebrate blood in Anopheles coluzzii and we reveal it can also be used to feed other anopheline species;
Supplementation of the blood-free diet with the vertebrate peptides that activate GPCRs for regulating reproduction and metabolism reveals that they modify mosquito physiology;
Of the tested human peptides, P2 (GLP 2), had the most notable effect when it was introduced in the blood-free artificial diet and it significantly increased VTG expression, mosquito egg production and offspring fitness relative to blood-fed mosquitoes.

P2 is a 33-amino acid peptide present in enteroendocrine L-cell and released in response to nutrient intake and it stimulates cell proliferation, inhibition of apoptosis and proteolysis in the small and large intestine in human. It belongs to a family of peptides with functions in the gastrointestinal (GI), carbohydrate metabolism and appetite regulation. The effect of human or other vertebrate GLP2 peptides in invertebrates has not yet been explored.

The use of the artificial fresh-blood-free meal herein described for mosquito rearing will help reduce costs, effort and eliminate live animal use for mosquito culture opening-up new opportunities for vector-borne diseases control.

### DESCRIPTION OF THE FIGURES

FIG. 1. Relative expression of vitellogenin precursor 24 hours post-peptide injection. The results represent the mean±SE of biological replicates from 3 independent experiments. Relative expression of Vg was determined using the ΔΔCT method using as the control female mosquitoes injected with PBS. P1 to P6 and P Mix represent the peptides tested and listed in Table 1.

FIG. 2a. Effects of the fresh-blood-free diets on oogenesis: Expression of vitellogenin precursor. Relative expression was determined using the ΔΔCT method as fold change to mosquitoes fed on the ILD, where RLD-NoP and ILD-NoP represent liquid diet compositions not supplemented with peptides, blood represent conventional blood diets and the remaining RLDs and ILDs represent respectively Rich Liquid Diet and Initial Liquid Diet supplemented with the correspondent indicated peptide P1 to P6.

FIG. 2b. Effects of the fresh-blood-free diets on oogenesis: Percent of mosquito females with eggs. Females were dissected 48 h post-feeding. The results represent the mean±SE of 3 biological replicates.

The diet composition abbreviations respective to the axis values have the same meaning as described before in FIG. 2a. ILD+/−P represents ILD compositions with and without supplementation of peptides, respectively. Asterisks indicate statistically significant (P<0.05)

FIG. 2c. Effects of the blood-free diets on oogenesis: Oocyte structure. Fluorescent confocal microscopy of oocytes stained with Nile red (lipids stain, red) and Dapi (nuclei stain, blue). Images were acquired with 20×, 40×, and 60× objectives using a Leica TCS SP5 laser scanning confocal microscope. Asterisks indicate statistically significant (P<0.05) results of an unpaired t test when compared to the blood-fed group (control). Blood represent conventional blood diets, and RLD and ILD represent respectively Rich Liquid Diet and Initial Liquid Diet.

FIG. 3. Feeding rate. The relative percent of fed and unfed females is indicated. Asterisks indicate groups significantly different (p<0.05 to p<0.0001) from the blood-fed control group and § indicates peptide supplemented RLD groups significantly different (p<0.0001) from the equivalent peptide enriched ILD (Fisher's exact test).

The diet composition abbreviations respective to the axis values have the same meaning as described in FIG. 2.

FIG. 4. Effect of fresh-blood-free meals on mortality and survival. After egg laying, mosquitoes were followed through their life cycle until the last pupae emerged and data was analysed against the number of eggs per female.

Progeny mortality for (a) Larvae, (b) Pupae and (c) Adults. Offspring's progeny survival represented as (d) Number of females produced in the different diet groups and (e) Number of males produced in the different diet groups.

Three independent experiments were performed, each consisting of 30 mosquitoes per diet (n=90/diet). Vertical bars represent Standard Error of the mean (SE). No significant differences were observed between the blood-fed group and non-blood fed groups using an unpaired t test.

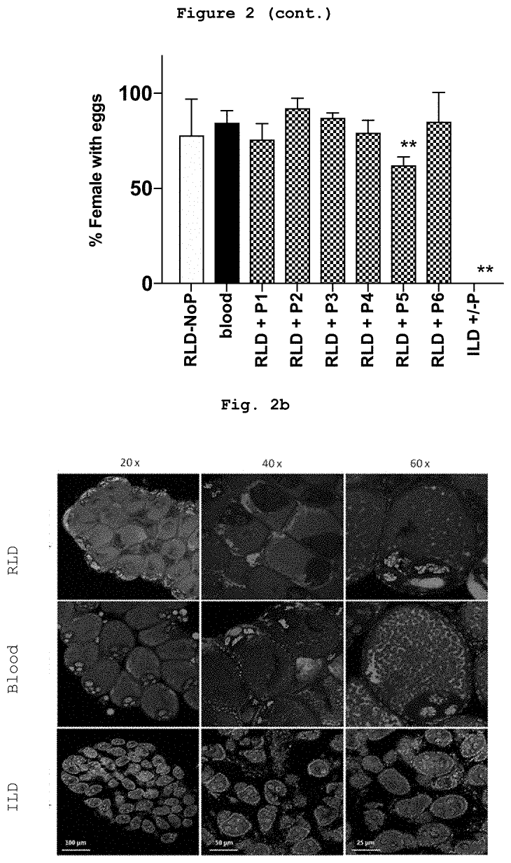

The diet composition abbreviations respective to the axis values have the same meaning as described in FIG. 2.

### GENERAL DESCRIPTION OF THE INVENTION

The present invention relates to blood-free-diets for malaria mosquito vectors comprising supplements of physiological concentrations of several different human GPCR ligands and/or other supplements such as a protein source, a phagostimulant and cholesterol. For this purpose, several artificial blood free meal formulations for female mosquitoes are herein disclosed.

These formulations were tested for mosquitoes egg stimulation, development and viability and their effect was compared with mosquitoes fed with conventional diets comprising vertebrate fresh blood showing that the diets of the invention stimulate oogenesis and egg production and has a similar or superior effect on mosquito fitness relative to a standard vertebrate blood.

### 1. Selection of Vertebrate GPCRs Peptide Candidates

Candidate molecules that circulate in human blood plasma and that stimulate different GPCRs were selected based on: a) their involved in the regulation of reproduction and nutrient metabolism in humans, and b) the identification of orphan GPCRs in the mosquito genome that are sequence orthologues of human receptors, as shown in Table 1 below.

Human ligands for GPCRs of several receptor families were selected and included peptides that activate Class A GPCRs (a.k.a Rhodopsin family GPCRs): oxytocin, galanin (GAL), kisspeptin, neuropeptide Y (NPY) that are basic determinants of reproductive functions, luteinizing hormone releasing hormone (LHRH), which stimulates the release of luteinizing hormone (LH) and follicle-stimulating hormone (FSH) and triggers ovulation in females and melatonin (MT) which regulates circadian rhythms of feeding and peaks in the blood at dawn, when mosquitoes are more actively searching a blood meal; Ligands of Class B GPCRs (a.k.a. Secretin-GPCRs) glucagon-like peptide 1 (GLP1), glucagon-like peptide 2 (GLP2) and vasoactive intestinal peptide (VIP) that regulate feeding behaviour, gut motility, glucose and insulin metabolism and parathyroid hormone (PTH), calcitonin (CT) and corticotrophin releasing hormone (CRH), which regulates ion metabolism and the stress response in humans and other vertebrates and also indirectly affects feeding behaviour; and of Class C GPCRs (a.k.a Glutamate GPCRs) the Glutamate and γ-aminobutyric acid (or GABA) which are two important neurotransmitters that stimulate feeding. Two fish peptides (CT and LHRH) were also tested as they are potent activators of the human peptide receptors and have similar functions to their human orthologues. So far, no potential activity or physiological effect of the selected human GPCR-ligands in mosquitoes is known. Table 1 presents the peptide description.

TABLE 1
List of the peptides of the invention and respective abbreviations

| Peptide | Abbreviation |
|---|---|
| Glucagon-like peptide 1 | P1 |
| Glucagon-like peptide 2 | P2 |
| Parathyroid hormone | P3 |
| Salmon calcitonin | P4 |
| Human calcitonin | P5 |
| γ-aminobutyric acid | P6 |
| Salmon Luteinizing Hormone Releasing Hormone | P7 |
| Human Luteinizing Hormone Releasing Hormone | P8 |
| Galanin | P9 |
| Vasoactive intestinal peptide | P10 |
| Glutamate | P11 |
| Oxytocin | P12 |
| Kisspeptin | P13 |
| Neuropeptide Y | P14 |
| Melatonin | P15 |
| Corticotropin-releasing hormone | P16 |
| Combination of peptides 1 to 6 | P Mix |

### 2. Blood-Free Diet Formulations

Several different blood-free diets were formulated for mosquito reproduction and egg development. The formulations of the invention comprise one or more peptides as listed in Table 1. These formulations may also comprise other supplements for enhancing the properties and effects on mosquitos of the diets of the invention, namely: (a) a protein source (b) a phagostimulant and (c) cholesterol.

These diets shown to be an effective artificial meal, free of blood that mimics a vertebrate blood meal and represents an important advance for the sustainability of Anopheles spp. mosquito rearing in captivity.

Peptides were added either to an initial liquid diet (ILD) providing amino acids, vitamins and carbohydrates, or to a rich liquid diet (RLD), a modification of the ILD supplemented with: (a) a source of proteins, such as bovine serum albumin (BSA), ovalbumin or lactalbumin, preferably BSA, which are essential for egg maturation, (b) a phagostimulant such as adenosine triphosphate (ATP), adenosine monophosphate (AMP), adenosine diphosphate (ADP), preferably ATP, and c) cholesterol.

Insects cannot synthesize cholesterol de novo and it is a precursor of the ecdysteroid hormone with a key role in yolk synthesis and egg maturation. Due to this fact, several concentrations of this compound in the diet compositions were tested.

Initial liquid diet (ILD) is based on DMEM tissue culture medium (Dulbecco's modified Eagle's medium) supplemented with the selected and listed peptides of the invention.

Table 2 show the major components of ILD and RLD base compositions, wherein the supplements of RLD added to the ILD are marked in bold and with *. Thus * means that the respective component is only present in RLD diets.

Base compositions are compositions not comprising the addition of the selected peptides of Table 1.

TABLE 2
Major components of ILD and RLD diets base
compositions with no addition of peptides

| Components | g/L |
|---|---|
| *Proteins | 1-500 |
| *Phagostimulant | 0.05-1.00 |
| *Cholesterol | 0.015-1.00 |
| Calcium chloride anhydrous | 0.2 |
| Choline chloride | 4.00E−03 |
| D-calcium pantothenate (vitamin B5) | 4.00E−03 |
| D-glucose anhydrous | 4.5 |
| Ferric nitrate nonahydrate | 1.00E−04 |
| Folic acid | 4.00E−03 |
| Glycine | 0.03 |
| I-inositol | 7.00E−03 |
| L-arginine monohydrochloride | 0.084 |
| L-cystine dihydrochloride | 0.063 |
| L-glutamine | 0.584 |
| L-histidine monohydrochloride | 0.042 |
| monohydrate | |
| L-isoleucine | 0.105 |
| L-leucine | 0.105 |
| L-lysine monohydrochloride | 0.146 |
| L-methionine | 0.03 |
| L-phenylalanine | 0.066 |
| L-serine | 0.042 |
| L-threonine | 0.095 |
| L-tryptophan | 0.016 |
| L-tyrosine disodium salt dihydrate | 0.104 |
| L-valine | 0.094 |
| Magnesium sulfate anhydrous | 0.098 |
| Niacinamide (nicotinamide) | 4.00E−03 |
| Phenol red | 0.015 |
| Potassium chloride | 0.4 |
| Pyridoxine Monohydrochloride | 4.00E−03 |
| Pyruvic acid sodium salt | 0.011 |
| Riboflavin (vitamin B2) | 4.00E−04 |
| Sodium bicarbonate | 3.7 |
| Sodium chloride | 6.4 |
| Sodium phosphate monobasic anhydrous | 0.109 |
| Thiamine amonohydrochloride (vitamin B1) | 4.00E−03 |

The protein source is bovine serum albumin (BSA), ovalbumin or lactalbumin in a concentration of 50-500 g/L of the final diet composition, preferably in a concentration of 100-300 g/L of the final diet composition, more preferably in a concentration of 150-250 g/L of the final diet composition. In a preferred embodiment, the protein source is BSA.

The phagostimulant is adenosine monophosphate (AMP), adenosine diphosphate (ADP) or adenosine triphosphate (ATP) in a concentration of 0.05-1.0 g/L of the final diet composition, preferably in a concentration of 0.1-0.75 g/L of the final diet composition, more preferably in a concentration of 0.5-0.6 g/L of the final diet composition. In a preferred embodiment, the phagostimulant is ATP.

The cholesterol is present in a concentration of 0.015-1.0 g/L of the final diet composition, preferably in a concentration of 0.1-0.5 g/L of the final diet composition, more preferably in a concentration of 0.05-0.25 g/L of the final diet composition.

Stock-compositions of the blood-free diets of the invention are thus obtained by supplementing the ILD and RLD diets of Table 2 with each one of the listed peptides of Table 1 (P1 to P16).

Other compositions are also provided by producing ILD and/or RLD supplemented with two or more of the peptides of Table 1, thus compositions having a mixture of peptides, or by mixing two or more stock compositions as described above.

Peptides are present in the compositions in several different concentrations varying in a range of 0.1-100 μM in the final concentration of the diet composition, preferably from 0.5 to 50 μM, more preferably 1.0 to 25 μM, even more preferably 2.5 to 12.5 μM, more advantageously 5 to 10 μM.

Therefore, four types of base diets were produced:
1. Non peptide supplemented ILD-COMPARATIVE;
2. Peptide supplemented ILD (S-ILD)-INVENTIVE;
3. Non peptide supplemented RLD-INVENTIVE; and
4. Peptide supplemented RLD (S-RLD)-INVENTIVE.

### 3. Selection of Vertebrate Peptide Candidates

### 3.1. Peptide Microinjections.

Peptides that circulate in human blood plasma and regulate reproduction and nutrient metabolism in humans were selected including two fish peptides that activate human receptors were screened in vivo for their capacity to trigger vitellogenin (Vg) transcription, a molecular marker of oogenesis initiation in the female mosquito fatbody. In nature the expression of this gene is triggered by blood meal and is fundamental for oogenesis and egg production.

Vitellogenin (Vg) transcription in the female mosquito fatbody was accessed by Quantitative Polymerase Chain Reaction. Vg transcript abundance was evaluated 24 hours post-feed, when maximal Vg expression is expected. The primers used in PCR reactions are listed in Table 3 below.

TABLE 3
Primers used for q-RT-PCR reactions

| Target | | Sequence 5′-3′ |
|---|---|---|
| S7 (AGAP010592) | Forward | GAGGTGGTCGGTATCC |
| | Reverse | CGATGGTGGTTTATCC |
| Vg (AGAP004203) | Forward | ACGAAAACCATGACCGCTCT |
| | Reverse | CTTGGGACGGATCACCAAAT |

PCR reactions included a standard curve, melting curves were performed to detect primer dimers and negative control reactions were included to assess for genomic contamination. PCR reaction efficiencies and r² (coefficient of determination) were calculated for each target gene and transcript expression was normalized using ribosomal S7 subunit as reference gene.

Two-day-old female Anopheles coluzzii mosquitoes were cold-anaesthetized and injected intrathoracically with each one of the peptides or mixtures listed in Table 1. For each experiment, a control group injected with PBS was included.

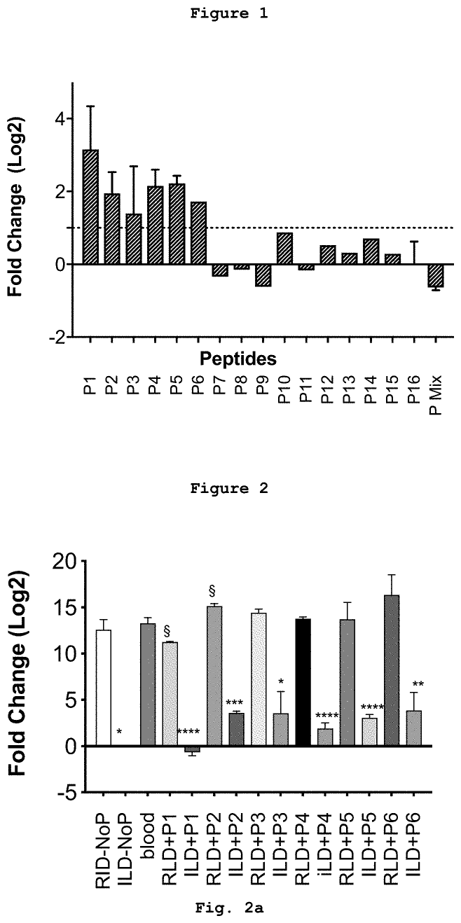

Of the selected vertebrate peptides tested and injected into the thorax of Anopheles coluzzii (previously known as Anopheles gambiae s.s. M form) only 6 (P1 to P6) induced up-regulation of Vg expression 24 h post-injection (FIG. 1) and their physiological effect on mosquito reproduction and fertility was subsequently tested.

### 4. Mosquitoes Feeding

For comparison purposes mosquitoes were fed ILD, RLD, S-ILD, S-RLD or on mouse blood as a golden standard. Mosquitoes were fed for approx. 60 min using a standard membrane feeding assay (MFA) and directedly on anesthetized mice.

4.1 Mosquitoes Feeding with the Diet Formulations of the Invention

Diets containing the different listed peptides of Table 1 and diet compositions of the invention, as described previously, were supplied to female mosquitoes using a standard artificial feeding apparatus, as the one described by Lopes, L. F. et al. (Lopes, L. F., Abrantes, P., Silva, A. P., Dorosario, V. E. & Silveira, H. Plasmodium yoelii: The effect of second blood meal and anti-sporozoite antibodies on development and gene expression in the mosquito vector, Anopheles stephensi. Exp. Parasitol. 115, 259-269. 2007).

Mosquitoes were kept inside paper cups covered with a net. Each cup had a glass feeder and a constant water flow supply. A thermoplastic film was placed at the mouth of the feeder and a pre-warmed meal was pipetted into the glass feeder. The selected peptides were thus tested at given concentration. Mosquitoes were allowed to feed for approximately 60 minutes. Unfed mosquitoes were removed, and fully engorged female mosquitoes were kept under adequate conditions.

### 4.2 Mosquitoes Feeding Directly on Mice

Female CD1 mice (Mus musculus) were intraperitoneally anesthetized and female mosquitoes were allowed to feed directly on healthy female CD1 mice for 30-45 min.

### 5. Oogenesis and Oocyte Structure

To test the potential involvement of the selected peptides on mosquito reproduction and egg development, different diets were formulated (as described above (section 1). ILD and RLD supplemented with peptides (S-ILD and S-RLD) were fed to mosquitoes using the standard MFA (section 4).

Oogenesis was assessed by changes in Vg expression 24 h post-feeding (FIG. 2a). Vg expression levels in the fatbodies was quantified using the ΔΔCT method a) after a blood meal, b) after microinjection with the different peptides, and c) after being fed on the various artificial diets as described in section 4.

To confirm that Vg expression induced oogenesis progression, the number of females presenting eggs was evaluated (FIG. 2b). The oocyte structure and lipid deposition were also evaluated by confocal microscopy. For this purpose, the ovaries of Anopheles coluzzii female mosquitoes were collected approximately twenty-four hours after feeding on blood or on supplemented a liquid diet having compositions according to the described above.

Except for the S-ILD, oocyte and ovary development of mosquitoes fed on S-RLD or blood diets was similar (FIG. 2b), and no morphological changes in ovary structure were observed (FIG. 2c).

The RLD, S-RLD and blood diets triggered similar Vg transcript levels showing that vitellogenesis occurred at a comparable rates in mosquitoes fed either on blood-free diets of the invention or fresh blood diet.

Of the blood-fed mosquitoes 85% produced eggs. Egg development occurred in 78% of the females fed on S-RLD and the highest percent of females with eggs was obtained for S-RLD+P2 (GLP2) and S-RLD+P3 (PTH) (FIG. 2b).

No egg development was observed with ILD, with or without peptide (S-ILD or ILD).

### 6. Feeding Rate

A successful artificial diet has to be as attractive to female mosquitoes as a fresh blood meal. Therefore, mosquito feeding success was investigated being the feeding success assessed by the numbers of engorged females.

Engorged females are defined as females with fully dilated abdomen and visible midgut content from the outside.

Groups of female mosquitoes fed with the different diets and the proportion of fully fed females were compared with the control blood-fed group.

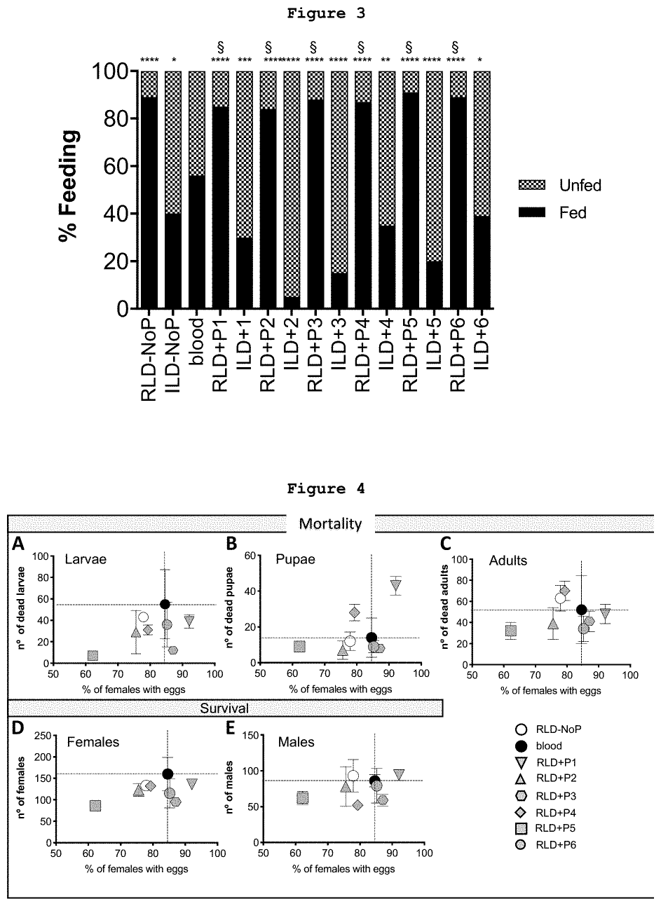

The percent of engorged female mosquitoes was always significantly higher in the RLD relative to the blood-fed group (FIG. 3) being the higher rates of feeding success, as assessed by the numbers of engorged females, obtained for the S-RLD. The highest rate of engorged females was obtained with the S-RLD supplemented with P5 (CT) diet.

Rates of engorgement with the RLD increased more than 20% relative to the blood-fed mosquitoes, showing that these diets are highly attractive to female mosquitoes when using artificial membrane feeding.

### 7. Egg Production, Fecundity and Fertility

The nutritional quality of each diet was evaluated by assessing the egg production, fecundity and fertility of female mosquitoes during the first gonotrophic cycle after artificial membrane feeding. By including fertility and fecundity as well as initiation of oogenesis it was possible to directly establish the effect of the diet on successful egg production or egg laying.

For that purpose, mosquitoes were fed with different diets, namely conventional blood diet and RLD diets with or without peptides supplement (RLD, S-RLD). As a proxy of feeding success, the number of mosquitoes that were fully engorged was recorded and the percent of fed mosquitoes determined.

To determine the number of eggs per female mosquitoes were dissected, the ovaries collected and the number of eggs per female determined under a magnifying glass.

For egg laying and mosquito development, fully engorged females were placed in individual cages with humidified filter paper for egg laying. Eggs were counted 48 and 72 h post-feed under magnifying glass. The total number of eggs/female was registered.

The data shows that oogenesis and egg production by mosquitoes fed with the formulations of the present invention was similar to the ones obtained by mosquitoes fed on blood.

In addition, the formulations of the present invention result in a better rate of number of eggs oviposited when compared with those of Sumba et. al. (Sumba, L. A. et al. Daily oviposition patterns of the African malaria mosquito Anopheles gambiae Giles (Diptera: Culicidae) on different types of aqueous substrates. J. Circadian Rhythms 2, 6. 2004), for Anopheles gambiae strain fed on the forearm of a human volunteer, in which the mean number of eggs oviposited was of 22.6±5.5 eggs/female.

### 8. Mosquito Fitness

Mosquito fitness is a determinant factor for the success of a mosquito colony when they are released into the wild. Colonies from each diet were maintained under standard insectary conditions, and the life cycle was followed for one generation. Larvae, pupae and adult mortality were recorded.

Offspring from females fed on S-RLD supplemented with P2 (GLP2), P3 (PTH) or P6 (GABA) showed better performances (eg. lower rates of dead larvae and dead adults/egg number/female) (FIG. 4a-c) when compared to blood and other diets.

However, the total number of female progeny was slightly lower than in progeny of blood-fed females (FIG. 4d-e). A blood meal had the highest impact on larvae mortality (FIG. 4b), showing that stable highly nutritional artificial diets, without fresh blood, can reduce mortality improving mosquito rearing success. Variability (SE) was always higher in the blood group (FIG. 4), when compared with other diets and is probably a reflection of the variable composition of blood and emphasises the usefulness of blood-free diets.

Except for the S-RLD supplemented with P5 (CT), the results obtained from the other dietary groups were similar to the blood fed colonies, showing a considerable advance over published studies (reviewed in Cosgrove, J. B. & Wood, R. J. Effects of variations in a formulated protein meal on the fecundity and fertility of female mosquitoes. Med. Vet. Entomol. 10, 260-4. 1996), in which artificial blood meals only showed relative success in Aedes mosquitoes, but were of limited or no-success when applied to Anopheles mosquitoes (in Gonzales, K. K. & Hansen, I. A. Artificial Diets for Mosquitoes. Int. J. Environ. Res. Public Health 13. 2016). Recently a successful plasma-based artificial meal was described for Anopheles mosquitoes (in Baughman, T. et al. A highly stable blood meal alternative for rearing Aedes and Anopheles mosquitoes. PLoS Negl. Trop. Dis. 11, e0006142. 2017) but lower feeding rates and estimated reproductive potential were obtained relative to the blood-fed control.

These results demonstrate that the S-RLD has a similar or superior effect on mosquito fitness relative to the standard vertebrate blood meal. Diet supplementation with vertebrate peptides affected different aspects of the female mosquito physiology and the progeny suggesting that they are also biologically active in the mosquito and interfere with reproduction and fitness.

### 9. Effect of the Artificial Diets on Another Anopheles Species Oogenesis

As a proof of principal and to test the capacity of the formulated blood-free diets to trigger egg development in other Anopheles species a RLD diet (with no peptide supplementation) was administrated to Anopheles stephensi and Anopheles aquasalis female mosquitoes using a similar procedure to that described above and egg production assessed 24 hours post-feeding. Developing eggs were identified in 70% to 93% of the Anopheles stephensi females and 33% to 35% of the Anopheles aquasalis females and the number of eggs per female was 53±10 and 46±27, respectively.

### 10. Statistical Analysis

Data are presented as the mean±standard deviation of at least three independent experiments (except where otherwise indicated), and the corresponding standard deviations in histograms are represented by error bars. The Student's t test was used to compare independent groups when data followed a Gaussian distribution, and differences were considered significant when P≤0.05. The Fisher's exact test was used to compare the differences on proportions among distinct diet-fed groups. The statistical analysis was performed on GraphPad Prism6 software.

### EXAMPLES

### General

### Assays and Tests

Except otherwise indicated, all reactions were performed at room temperature (+/−20° C.), reagents were purchased from Sigma-Aldrich Corporation (St. Louis, Mo., USA), and female mosquitoes of Anopheles coluzzii (former Anopheles gambiae M form) Yaounde strain were used.

The peptides calcitonin (from human), calcitonin (from salmon), glucagon-like peptide 1 (1-37) (human, bovine, guinea pig, mouse, rat) trifluoroacetate salt, and glucagon-like peptide 2 (1-33) (human) ammonium acetate salt were purchased from Bachem (Germany).

The peptides parathyroid hormone (from human), oxytocin (from human), kisspeptin 10 (from human), melatonin, and neuropeptide Y (from human, rat) were purchased from Tocris (Biogene, Spain).

Mosquitoes were obtained from a laboratory colony of Anopheles coluzzii (Yaoundé line) and Anopheles stephensi. Anopheles aquasalis were obtained from a colony at the Entomology Department Insectary of the Fundação de Medicina Tropical Dr Heitor Vieira Dourado (FMT-HVD) (id approvals: CEUA 01/2013), that were derived from a colony established in 1995 (in Cardozo Goncalvez De Carvalho, S. et al. Temperature Influence on Embryonic Development of Anopheles albitarsis and Anopheles aquasalis. Mem Inst Oswaldo Cruz Rio Janeiro 97, 1117-11120. 2002).

### Mosquitoes Maintenance

Mosquitoes were maintained under standard insectary conditions of 26±1° C., 75% humidity and a 12 h:12 h light:dark cycle. Adult mosquitoes were fed on 10% glucose solution ad libitum until the day before feeding trials.

### Feeding of Mosquitoes

From Conventional Blood-Diets

Female CD1 mice (Mus musculus), obtained from the IHMT Animal house, were intraperitoneally anesthetized with 20% Imalgène 1000 (Merial, Portugal) and 10% Rompun (Bayer, Portugal). Female mosquitoes fed directly on healthy female CD1 mice for 30-45 min, with regular monitoring to verify that mice were anesthetized. Unfed mosquitoes were removed and fully engorged mosquitoes were kept at 26±1° C., 75% humidity. When needed, a cardiac puncture was performed on anesthetized mouse to collect 1 mL of blood for artificial blood feeding assays.

From Blood-Free Diets

Mosquitoes (n=30 approximately) were kept inside paper cups covered with a net. Each cup had a glass feeder on top connected to 2 plastic tubes for water inlet and outlet and temperature within the multiple cylindrical water-jacked plastic was kept at 37.5° C. by a constant water flow supply. Parafilm® was stretched across the mouth of the feeder and 1 mL of a pre-warmed meal was pipetted into the glass feeder. Mosquitoes were allowed to feed for 60 minutes. Unfed mosquitoes were removed and fully engorged female mosquitoes were kept at 26±1° C. under 75% humidity.

### Fluorescence Confocal Microscopy

The ovaries of Anopheles coluzzii female mosquitoes were collected twenty-four hours after feeding on blood or on supplemented liquid diet and fixed for 15 min with 4% v/v paraformaldehyde (Alfa Aesar, Massachusetts, USA) in PBS. Samples were washed twice with 0.5% Triton in PBS and incubated with 250 μM Nile Red (MP Biomedicals, USA) for one hour in the dark. Samples were washed twice with 0.5% Triton in PBS and the slides were mounted by using Vectashield with dapi (Vector Laboratories, USA) and analysed using a Leica TCS SP5 laser scanning confocal microscope (20×, 40×, and 60× objectives).

RBA Extractions & cDNA Synthesis

Total RNA from female fatbodies was isolated using the TRI Reagent protocol and treated with 1 U DNase for 1 min according to the manufacturer's instructions to eliminate genomic DNA contamination. DNase I treated total RNA (1.5 μg) was denatured at 70° C. for 10 min, quenched on ice for 5 min and used for cDNA synthesis in a 20 μl final reaction volume containing 10 μL of 2× RT buffer mix, 1 μL of 20× RT enzyme mix (Thermofisher, Alfagene, Portugal), and nuclease-free water. cDNA was synthesized for 60 min at 37° C. followed by 5 min at 95° C. to stop the reaction and hold at 4° C.

The quality and quantity of the cDNA produced was assessed by PCR amplification of the ribosomal protein S7 unit using the following protocol: 95° C. for 3 min; 35 cycles of 95° C. for 30 sec, 60° C. for 30 sec, 72° C. for 30 sec, followed by 72° C. for 5 minutes (in Félix, R. C. & Silveira, H. The Interplay between Tubulins and P450 Cytochromes during Plasmodium berghei Invasion of Anopheles gambiae Midgut. PLoS One 6, e24181. 2011). PCR reactions were carried out for a 10 μl final reaction volume containing 1.5 mM MgCl₂ (Thermo Scientific, Alfagene, Portugal), 0.2 mM dNTPs (GE Healthcare, Spain), 0.25 μM of each gene specific primer pair and 0.5 U of DreamTaq DNA Polymerase (5 U/μl, Thermo Scientific, Alfagene, Portugal) and the amplification products analysed on agarose electrophoresis gel.

### Quantitative Polymerase Chain Reaction

Quantitative Real-time Polymerase Chain Reaction (q-RT-PCR) analysis was used to quantify the expression of Vitellogenin-1 precursor (Vg), a protein biomarker of mosquito oogenesis initiation. Vg expression levels in the fatbodies were quantified using the ΔΔCT method a) after a blood meal, b) after microinjection with the different peptides, and c) after being fed on the various artificial diets. Primers used are described in Table 3 as presented earlier. The amplification was performed at 64° C. for S7 and 60° C. for Vg.

Briefly, cDNA samples were diluted (1:10 or 1:5) with ultrapure water prior to use as a template in q-RT-PCR and reactions were performed in triplicate (<5% variation between replicates) using a CFX Connect Real-Time PCR Detection System (Bio-Rad, Portugal) for 96-well microplates (Bio-Rad, Portugal). Analyses were performed in 20 μl final reaction volume with 300 nM of forward and reverse primer, SsoFast EvaGreen supermix (Bio-Rad, Portugal) and 2 μl of the diluted cDNA template. Optimized cycling conditions consisted of 95° C. for 30 sec, followed by 45 cycles of 95° C. for 5 sec and the appropriate annealing temperature for each primer pair for 10 sec. PCR reactions included a standard curve, melting curves were performed to detect primer dimers and negative control reactions were included to assess for genomic contamination. PCR reaction efficiencies and r² (coefficient of determination) were calculated for each target gene and transcript expression was normalized using ribosomal S7 subunit as reference gene.

### Peptide Microinjections

Two-day-old female Anopheles coluzzii mosquitoes were cold-anaesthetized and injected intrathoraxically with 69 nL of 10 μM peptide. For each experiment, a control group injected with PBS was included. Injections were performed using a microinjection system (Nanoject; Drummond Scientific). The complete list of the peptides used and respective abbreviations are presented on Table 1 presented earlier.

### Tissue Collection

Fatbodies were collected from pools of 30-35 mosquitoes. Tissues were dissected, transferred to RNAlater (Ambion, Alfagene, Portugal) and stored at −20° C. until RNA extraction. For the blood meal assays, mosquito fatbodies were collected at different time points (3, 6, 12, 24, 28, and 32 hours post blood meal). Fatbodies from females feed on 10% glucose at the same time points were used as controls. For the peptides screening experiment and artificial diet assays, mosquito fatbodies were dissected 24 h post-injection and 24 h post-feeding, respectively.

### Fecundity, Fertility and Adult, Pupae and Larvae Development and Mortality

Experimental feeding of mosquitoes using blood or artificial blood-free diets was performed as described above. As a proxy of feeding success, the number of mosquitoes that were fully engorged was recorded and the percentage of fed mosquitoes determined. To determine the number of eggs per female, at 48 h post-feed, mosquitoes were dissected, the ovaries collected and the number of eggs per female determined under a hand held magnifying glass. For egg laying and mosquito development, 30 fully engorged females were placed in individual cages (20×20×20 cm) with humidified filter paper for egg laying (30 females/replicate, 3 replicates/diet). Eggs were counted 48 and 72 h post-feed under a hand held magnifying glass. The total number of eggs/female was registered.

Eggs were collected into a water tray (23×15×6 cm) and mosquito development was followed until all pupae have emerged, for each diet. After hatching, a strict similar larval feeding regime was applied to all trays, using grounded fish food (1:1 ASTRA pond-fish-sticks and TetraMin flacks). Trays were fed approximately with 13 mg every day. Water levels were kept constant throughout the experiment by addition of dH₂O. Pupae and larvae (L3 and L4) mortality was checked daily and dead stages were removed. Although there was abundant food, the dead larvae could have been cannibalized. As the larvae resulting from females fed with different diets were in similar conditions, we assume that if it occurs, cannibalism would be similar in all groups. The number of male and female adults for each diet was recorded by the end of the experiment when all pupae have developed into adult mosquitoes. Dead adults (females and males) were counted and removed daily. Adults were maintained on water with 10% glucose solution ad libitum until the day they died. The experiment ended when all pupae developed into adult mosquitoes in all diets. Three independent replicates were performed for each diet.

### Example 1. Preparation of the Base Liquid Diet Compositions

Two different blood-free diets were formulated, the base diets ILD and RLD, having the composition as described in Table 2 (section 2).

The ILD is a Dulbecco's modified Eagle's medium (high glucose with L-glutamine). RLD composition was the result of the addition of 0.55 g/L ATP, 1 g/L cholesterol, and 200 g/L BSA to ILD. Diets were prepared in a flow laminar chamber to keep all stock solutions sterile. All ingredients were thoroughly mixed, and filter sterilized (0.45 μm pore). For each experiment, diet formulae were freshly prepared from stock solutions. ILD and RLD compositions were used for comparative purposes.

### Example 2. Preparation of Liquid Diet Compositions Supplemented with Peptides

For the purpose of investigating mosquito reproduction and egg development several different diets were formulated comprising the selected peptides of Table 1 (section 1). Peptides were added either to an initial liquid diet (ILD) or to a rich liquid diet (RLD), as described in Example 1, in a concentration of 10 μM in the final concentration of the diet composition, thus resulting in the S-ILD and S-RLD blood free liquid diets.

### Example 3. Mosquito Feeding Rate Assessment

Mosquito feeding success was investigated by the numbers of engorged females fed with two different base diets (ILD and RLD), two peptide supplemented diets (S-ILD and S-RLD), as described in Examples 1 and 2, and compared with conventional mouse blood fed mosquitoes.

The percent of engorged female mosquitoes was always significantly higher in the RLD fed group relative to the blood-fed group (FIG. 3). Approximately 60% of the blood-fed females were fully engorged relative to 40% of the mosquitoes fed with the ILD.

The highest rate of feeding success, as assessed by the numbers of engorged females, was obtained for the S-RLD (84-91%). The highest rate of engorged females was obtained with the S-RLD supplemented with P5 (CT) diet (91.2%±6.8).

### Example 4. Oogenesis and Oocyte Structure

Oogenesis was assessed by changes in Vg expression 24 h post-feeding mosquitoes with the artificial diets of Examples 1 and 2 and conventional vertebrate blood diet (FIG. 2a). To confirm that Vg expression induced oogenesis progression, the number of females presenting eggs was evaluated (FIG. 2b). Forty-eight hours post-feed mosquitoes were cold anesthetized, briefly washed in 70% ethanol and dissected under a magnifying glass on a drop of PBS. The ovaries were collected to determine the number of eggs per female.

Of the blood-fed mosquitoes 85% produced eggs. Egg development occurred in 78% of the females fed on S-RLD and the highest percent of females with eggs (92% and 87%) was obtained for S-RLD+P2 (GLP2) (p=0.042) and S-RLD+P3 (PTH) (FIG. 2b).

### Example 5. Egg Production, Fecundity and Fertility

Fully engorged mosquitoes fed with the blood-free diets RLD, S-RLD and conventional blood diets as described previously were maintained under optimal conditions and egg laying observed approx. 48 hours post-feeding. The egg number was counted 72 hours post-feeding before they were flooded with distilled water. Results are presented in Table 4 below.

TABLE 4
Egg batches produced by Anopheles coluzzii females

| TYPE OF | Total Egg Number | Eggs/Female |
|---|---|---|
| DIET | (±SE) | (±SE) |
| BLOOD diet | 7331330 | 24111 |
| RLD | 7631164 | 2515 |
| RLD + P1 | 7741343 | 26111 |
| RLD + P2 | 648158 | 2211 |
| RLD + P3 | 719 ± 82 | 25 ± 2 |
| RLD + P4 | 492 ± 62 | 16 ± 2 |
| RLD + P5 | 309 ± 18 | 10 ± 0 |
| RLD + P6 | 656 ± 57 | 22 ± 2 |

Females that were fed on fresh vertebrate blood, laid an average of 24 (±11) eggs whereas those fed on S-RLD laid on average 25 (±5) eggs per female. The best egg laying rates were achieved with S-RLD supplemented with P1 (GLP1) (26 (±11) eggs per engorged female). Females fed on S-RLD supplemented with P5 (CT) laid the lowest average number of eggs (n=10) and this was in line with the significant reduction in percentage of egg development recorded (FIG. 2b).

### Example 6. Effect of Cholesterol in the Feeding Rate and Egg Production

The effect of cholesterol in the feeding rate and egg production was assessed by supplementing different concentrations of this compound, namely 0.96 g/L, 0.24 g/L, 0.06 g/L, 0.015 g/L to RLD compositions having 0.55 g/L ATP, and 200 g/L BSA. Each assay was performed in triplicate.

The feeding rate was evaluated as described previously in Example 3. Results show no significant differences in the feeding rates where cholesterol was supplemented at a concentration as indicated above. However, the best results were obtained with RLD compositions having 0.24 g/L (88.9%). The number of eggs produced by each female was evaluated as described previously in Example 5 being the best results obtained with RLD compositions having a cholesterol concentration of 0.06 g/L (21.52 eggs/female) and 0.24 g/L (22.978 eggs/female).

1. A liquid artificial blood-free diet composition for rearing mosquitoes having an adequate amount of amino acids, vitamins and carbohydrates comprising:
one or more peptides selected from the group of Glucagon-like peptide 1 (P1), Glucagon-like peptide 2 (P2), Parathyroid hormone (P3), Salmon calcitonin (P4), Human calcitonin (P5), γ-aminobutyric acid (P6), Salmon Luteinizing Hormone Releasing Hormone (P7), Human Luteinizing Hormone Releasing Hormone (P8), Galanin (P9), Vasoactive intestinal peptide (P10), Glutamate (P11), Oxytocin (P12), Kisspeptin (P13), Neuropeptide Y (P14), Melatonin (P15) and Corticotropin-releasing factor (P16); and/or
a phagostimulant, a protein source and cholesterol.

2. A liquid artificial blood-free diet composition according to claim 1, wherein the selected peptides are present in the composition in several different concentrations varying in a range of 0.1-100 μM in the final concentration of the diet composition, preferably from 0.5 to 50 μM, more preferably 1.0 to 25 μM, even more preferably 2.5 to 12.5 μM, more advantageously 5 to 10 μM.

3. A liquid artificial blood-free diet composition according to claim 1, wherein the selected peptides present in the composition are Human Glucagon-like peptide 1 (P1), Human Glucagon-like peptide 2 (P2), Human Parathyroid hormone (P3), Salmon calcitonin (P4), Human calcitonin (P5) and/or γ-aminobutyric acid (P6).

4. A liquid artificial blood-free diet composition according to claim 1, wherein:
the protein source is bovine serum albumin (BSA), ovalbumin or lactalbumin in a concentration of 50-500 g/L of the final diet composition, preferably in a concentration of 100-300 g/L of the final diet composition, more preferably in a concentration of 150-250 g/L of the final diet composition;
the phagostimulant is adenosine monophosphate (AMP), adenosine diphosphate (ADP) or adenosine triphosphate (ATP) in a concentration of 0.05-1.0 g/L of the final diet composition, preferably in a concentration of 0.1-0.75 g/L of the final diet composition, more preferably in a concentration of 0.5-0.6 g/L of the final diet composition; and
the cholesterol is present in a concentration of 0.015-1.0 g/L of the final diet composition, preferably in a concentration of 0.1-0.5 g/L of the final diet composition, more preferably in a concentration of 0.05-0.25 g/L of the final diet composition

5. A liquid artificial blood-free diet composition according to claim 1 wherein:
the Human Glucagon-like peptide 1 (P1), Human Glucagon-like peptide 2 (P2), Human Parathyroid hormone (P3), Salmon calcitonin (P4), Human calcitonin (P5) and/or γ-aminobutyric acid (P6) peptides, alone or in combination, are present in the composition in several different concentrations varying in a range of 0.1-100 μM in the final concentration of the diet composition, preferably from 0.5 to 50 μM, more preferably 1.0 to 25 μM, even more preferably 2.5 to 12.5 μM, more advantageously 5 to 10 μM;
the protein source is bovine serum albumin (BSA) in a concentration of 50-500 g/L of the final diet composition, preferably in a concentration of 100-300 g/L of the final diet composition, more preferably in a concentration of 150-250 g/L of the final diet composition;
the phagostimulant is adenosine triphosphate (ATP) in a concentration of 0.05-1.0 g/L of the final diet composition, preferably in a concentration of 0.1-0.75 g/L of the final diet composition, more preferably in a concentration of 0.5-0.6 g/L of the final diet composition; and
the cholesterol is present in a concentration of 0.015-1.0 g/L of the final diet composition, preferably in a concentration of 0.1-0.5 g/L of the final diet composition, more preferably in a concentration of 0.05-0.25 g/L of the final diet composition.

6. A method for rearing mosquitos by feeding said mosquitoes with a liquid artificial blood-free diet composition as described in claim 1.

7. A method according to claim 6, wherein the liquid artificial blood-free diet composition is provided to the mosquitoes, during at least 30 minutes, preferably at least 60 minutes, by an artificial feeding apparatus as a pre-warmed meal.

8. A method according to claim 6 wherein the mosquitos to be reared are selected from the genus Aedes or from the genus Anopheles, preferably are mosquitoes from the genus Anopheles.

9. A process for producing a liquid artificial blood-free diet composition by supplementing the Dulbecco's modified Eagle's medium with one or more of the peptides listed in claim 1 and/or a phagostimulant, a protein source and cholesterol.

10. A process according to claim 9, wherein:
the selected peptides are present in the composition in several different concentrations varying in a range of 0.1-100 μM in the final concentration of the diet composition, preferably from 0.5 to 50 μM, more preferably 1.0 to 25 μM, even more preferably 2.5 to 12.5 μM, more advantageously 5 to 10 μM; and
the protein source is bovine serum albumin (BSA), ovalbumin or lactalbumin in a concentration of 50-500 g/L of the final diet composition, preferably in a concentration of 100-300 g/L of the final diet composition, more preferably in a concentration of 150-250 g/L of the final diet composition;
the phagostimulant is adenosine triphosphate (ATP) in a concentration of 0.05-1.0 g/L of the final diet composition, preferably in a concentration of 0.1-0.75 g/L of the final diet composition, more preferably in a concentration of 0.5-0.6 g/L of the final diet composition; and
the cholesterol is present in a concentration of 0.015-1.0 g/L of the final diet composition, preferably in a concentration of 0.1-0.5 g/L of the final diet composition, more preferably in a concentration of 0.05-0.25 g/L of the final diet composition.

11. The use of one or more peptides selected from the group comprising Glucagon-like peptide 1 (P1), Glucagon-like peptide 2 (P2), Parathyroid hormone (P3), Salmon calcitonin (P4), Human calcitonin (P5), γ-aminobutyric acid (P6), Salmon Luteinizing Hormone Releasing Hormone (P7), Human Luteinizing Hormone Releasing Hormone (P8), Galanin (P9), Vasoactive intestinal peptide (P10), Glutamate (P11), Oxytocin (P12), Kisspeptin (P13), Neuropeptide Y (P14), Melatonin (P15) and Corticotropin-releasing factor (P16) for producing a liquid artificial blood-free diet composition for rearing mosquitoes.

12. The use of one or more peptides according to claim 11, wherein the mosquitoes are from the genus Aedes or from the genus Anopheles, preferably the mosquitoes are from the genus Anopheles.
